(19) United States
(12) Patent Application Publication
(10) Pub. No.: US 2011/0142903 A1
(43) Pub. Date: Jun. 16, 2011

(54) CONTROLLED-RELEASE, INTRA-ARTICULAR THERAPEUTIC AGENT DELIVERY COMPOUND, AND A METHODOLOGY FOR THE CONTROLLED-RELEASE OF AN INTRA-ARTICULAR THERAPEUTIC AGENT DELIVERY COMPOUND

(76) Inventor: Nicholas Abidi, Scotts Valley, CA (US)

(21) Appl. No.: 12/639,711

(22) Filed: Dec. 16, 2009

(51) Int. Cl. A61K 9/00; A61P 29/00

(52) U.S. Cl. 424/423

(57) ABSTRACT

An intra-articular therapeutic agent delivery compound for controlled delivery of an intra-articular therapeutic agent to an intra-articular site. An intra-articular therapeutic agent. A substrate configured to bind with the intra-articular therapeutic agent, to be delivered to the intra-articular site, and to be distributed over a period of time thus releasing the intra-articular therapeutic agent.

### FIELD OF THE INVENTION

Embodiments in accordance with the present invention pertain to surgical procedures including but not limited to intra-articular procedures.

### BACKGROUND

Some current intra-articular or arthroscopic surgical procedures lead to an undesirable inflammatory side effect. The inflammatory side effect is typically found within the synovial membrane, known as the joint capsule, which makes the joint water tight or waterproof. The joint capsule is penetrated during an intra-articular surgical procedure when intra-articular instruments are placed into the joint. Commonly, the inflammatory side effect causes pain and inflammation to various tissues and leads to limited range of motion and mobility and to compromised usage of the joint. Such inflammatory side effects can result in a delay in return to normal function for the patient.

Conventionally, anti-inflammatory drugs (e.g. orally consumed NSAIDs, non-steroidal anti-inflammatory drugs) are used peri-operatively (both pre-operatively and post-operatively) to limit inflammation. Additionally, anti-inflammatory drugs may be introduced at the site during the intra-articular surgical procedure. Unfortunately, conventional application and use of anti-inflammatory drugs often does not provide adequate results. For example, patients using convention, anti-inflammatory drugs such as NSAIDs may experience side effects. Such side effects may also be in the form of a broad effect which is systemic. This is due to the fact that orally-consumed NSAIDs are not tissue specific, and that the patient's plasma concentration must be elevated in order to achieve a local effect.

### BRIEF DESCRIPTION OF THE DRAWINGS

The accompanying drawings, which are incorporated in and form a part of this specification, illustrate and serve to explain the principles of embodiments in conjunction with the description. Unless specifically noted, the drawings referred to in this description should be understood as not being drawn to scale.

FIG. 1 is a block diagram of an example intra-articular site with an application device for applying an intra-articular therapeutic agent delivery compound, in accordance with an embodiment.

FIG. 2 is a block diagram of an intra-articular therapeutic agent delivery compound, in accordance with an embodiment.

FIG. 3 is a flowchart of a method for forming an intra-articular therapeutic agent delivery compound for controlled delivery of an intra-articular therapeutic agent to an intra-articular site and for controlled delivery of an intra-articular therapeutic agent to an intra-articular site, in accordance with embodiments of the present invention.

### DESCRIPTION OF EMBODIMENTS

Reference will now be made in detail to various embodiments, examples of which are illustrated in the accompanying drawings. While the subject matter will be described in conjunction with these embodiments, it will be understood that they are not intended to limit the subject matter to these embodiments. Furthermore, in the following description, numerous specific details are set forth in order to provide a thorough understanding of the subject matter. In other instances, well-known methods, procedures, objects, and circuits have not been described in detail as not to unnecessarily obscure aspects of the subject matter.

### Overview of the Present Technology

As an overview, embodiments in accordance with the present technology provide a controlled-release, intra-articular therapeutic agent delivery compound, and a methodology for the controlled-release of an intra-articular therapeutic agent delivery compound. In one embodiment of the present technology, a controlled-release anti-inflammatory drug is introduced to an intra-articular site and the anti-inflammatory medication is slowly released into the site over a period of time (e.g. days or weeks).

Embodiments of the present technology provide controlled-release drug delivery devices and methods for using such devices. In one embodiment, the device comprises a drug core surrounded by a substantially impermeable polymeric outer layer which has one or more orifices through which body fluids may enter to dissolve the drug. The present technology is particularly appropriate for drugs that are very active even in extremely small quantities and whose sustained long-term administration is sought.

Various embodiments of the present technology have the ability to be implanted in a number of locations in a human or an animal to obtain localized or systemic effects of the drug that is released from the device. In one embodiment, the device is implanted in an intra-articular site to treat or prevent a variety of conditions such as bacterial and viral infections, inflammation, hemorrhage, etc.

### Controlled-Release Intra-articular Therapeutic Agent Delivery Compound

Arthroscopy of various joints throughout the body is an essential part of orthopedic surgical practice. Arthroscopy is mostly for therapeutic purposes (surgical reconstruction), but can also be for diagnostic purposes. Arthroscopy has reduced the traumatic nature of invasive joint surgery, which previously involved arthrotomy. Despite the les invasive nature of arthroscopy, post-operative excessive inflammation and bleeding can lead to arthrofibrosis and pain that extends post-operative recovery. Recently, the use of oral non-steroidal anti-inflammatory drugs (NSAIDs) peri-operatively has reduced morbidity associated with some of the surgical procedures. The unpredictable and undesirable mature of the systemic side effects of oral medication makes the oral medication route for NSAIDs less than desirable. Ideally, during a surgical procedure, when the operative surgeon had direct access to the pathological joint, it would be desirable to deliver a medication that would have a beneficial effect locally to reduce post-operative pathological inflammation and resultant disability. It has recently become acceptable during joint surgery to inject NSAID or steroidal medication into native and artificial joints during the operative procedure to reduce post-operative morbidity and expedite recovery. However, this method results in a one time dose of medication which has short acting results. This may not ideally match the patient's inflammatory cycle and have excessive early limitation of inflammation or insufficient limitation of inflammation at the desirable seven to thirty day time point post-operatively. In addition, the pathophysiology of the effect of the medication on the intra-articular tissues has not been studied.

Embodiments in accordance with the present invention provide a controlled-release, intra-articular therapeutic agent delivery compound that over time (i.e. in a controlled-release manner) will deliver a conventional medication without having to change the chemistry of the drug itself. The drug would then be gradually released over time. Embodiments of the present technology provide advantages over the current use of immediate release steroids at an intra-articular site peri-operatively during arthroscopy. Such current uses are rare and experimental.

In one embodiment, the present technology is comprised of an intra-articular therapeutic agent which is bound to a substrate. The intra-articular therapeutic agent is bound to the substrate in a manner such that the binding of the intra-articular therapeutic agent substrate will weaken over time and thereby release the intra-articular therapeutic agent from the substrate. Hence, when introduced to the intra-articular site (e.g. within the synovial capsule), the intra-articular therapeutic agent is slowly (e.g. in a controlled-released manner) provided to the intra-articular site. In one embodiment, the intra-articular site is an arthroscopic surgical site.

It should be appreciated that the intra-articular therapeutic agent may be, but is not limited to, a conventional drug or medication, a plurality of drugs, an anti-inflammatory drug, a non-steroidal anti-inflammatory drug, an analgesic drug, steroidal drug, dexamethasone, or a combination thereof.

In one embodiment, the intra-articular therapeutic agent is bound to a substrate such as an acrylonitrile butadiene styrene (ABS) resin or polymer such as PGLA-Novador. Although such specific materials are cited above for use as the substrate, embodiments in accordance with the present technology are also well suited to binding the anti-inflammatory drug with various other substrate materials. Furthermore, although embodiments in accordance with the present technology specifically refer to binding of an anti-inflammatory drug (e.g. a non-steroidal) to the substrate, various embodiments of the present technology are also well suited to binding other agents (e.g. a pain medication(s)) to the aforementioned or other types of substrates. Hence, a controlled-release of a pain medication or medications is achieved. Additionally, embodiments in accordance with the present technology are also well suited to binding a plurality of drugs to the substrate. In so doing, a controlled-release of multiple drugs is achieved.

In one embodiment, once the intra-articular therapeutic agent and substrate are bound together into a delivery compound, the delivery compound is applied an intra-articular site. The delivery compound may be delivered peri-operatively meaning that the delivery compound may applied to the intra-articular site before, after or during a procedure. In one embodiment, the delivery compound is in a liquid format. In other words, the delivery compound is microscopic and may be in a fluid, but does not precipitate so it stays in solution after application to the intra-articular site.

It should be appreciated that arthroscopy includes a minimally invasive surgical procedure in which an arthroscope or endoscope is inserted in a small incision to examine and treat a joint. It should be appreciated that intra-articular sites may include, but are not limited to, joints such as the knee, shoulder, elbow, wrist, hip, ankle, great toe, etc.

Benefits of the present technology include the possibility of the patient being able to begin work three days after a procedure. Also, negative side effects, including nausea, increased bleeding, bruising and scarring, associated with traditional treatment plans such as the injection and oral intake of narcotics is alleviated or eliminated by the present technology.

The intra-articular therapeutic agent delivery compound can be delivered to the intra-articular site with a variety of techniques. In one embodiment, the intra-articular therapeutic agent delivery compound is added to a fluid that is running through the intra-articular site during a procedure. For example, the fluid running through the intra-articular site may be lactadid ringers or normal saline. By adding the intra-articular therapeutic agent delivery compound to such a fluid, the intra-articular site is bathed in the intra-articular therapeutic agent delivery compound during the procedure and will limit the amount of inflammation during the procedure. The present technology would provide for a slow gradual delivery, similar to how antibiotics are used to treat an infected wound. In one embodiment, a portion of the intra-articular therapeutic agent delivery compound is deposited or applied to the intra-articular site before closing the intra-articular site. In one embodiment, 5-10 ccs of the intra-articular therapeutic agent delivery compound is deposited before closing. In one embodiment, the intra-articular therapeutic agent delivery compound may be applied or delivered to the intra-articular site via an injection using a syringe or hypodermic needle. Such an injection may be performed before, during or after a procedure. In one embodiment, the intra-articular therapeutic agent delivery compound may be applied or delivered to the intra-articular site via Lactated Ringer's solution, a solution that is administered intravenously and is isotonic with blood.

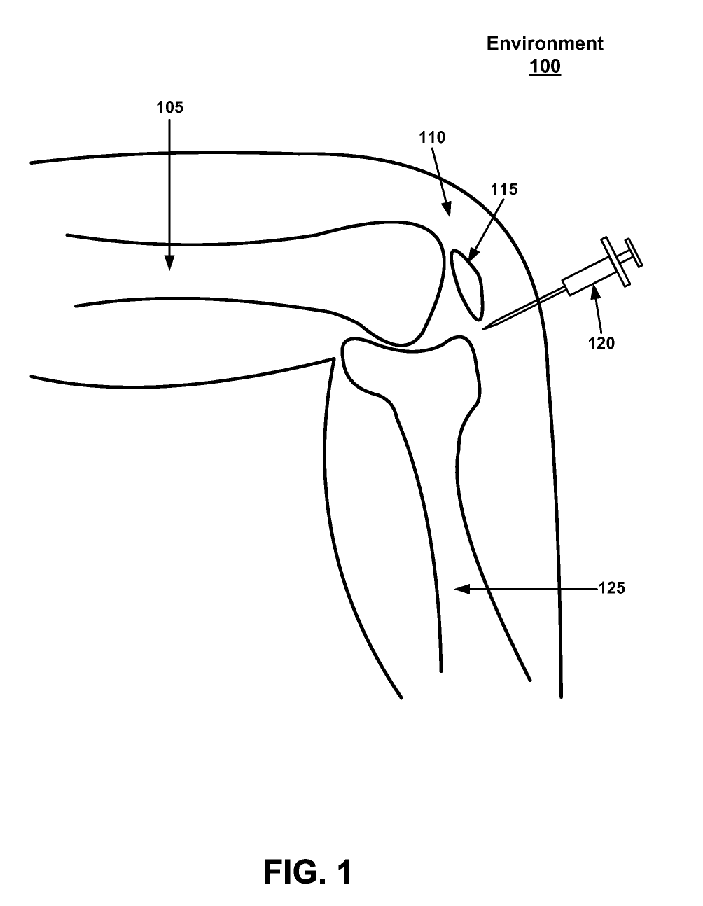

With reference now to FIG. 1, an illustrative diagram of an intra-articular site in accordance with embodiments of the present technology. In particular, FIG. 1 illustrates intra-articular site 110 and application device 102. It should be appreciated that while FIG. 1 generally depicts a knee, it is not meant to limit the present technology to one type of site nor one type of application device.

In one embodiment, FIG. 1 includes intra-articular site 110, femur 105, patella 115, tibia 125 and application device 120. FIG. 1 uses femur 105, patella 115, and tibia 125 to depict the femur, patella and tibia of a human body. FIG. 1 is drawn to illustrate application device 120 as a syringe used to inject the intra-articular therapeutic agent delivery compound into intra-articular site 110. It should be appreciated that application device 120 is not limited to a syringe, but may be another device used to apply a liquid compound to an intra-articular site. Application device 120 may be different device depending on the stage in which the intra-articular therapeutic agent delivery compound is applied. For example, a syringe may be used for application post-operatively while a different device is used during a surgical procedure. It should also be appreciated that the depiction of application device 120 being inserted into intra-articular site 110 is not drawn to represent an actual injection site but is merely represents application device 110 injecting the intra-articular therapeutic agent delivery compound at or near intra-articular site 110.

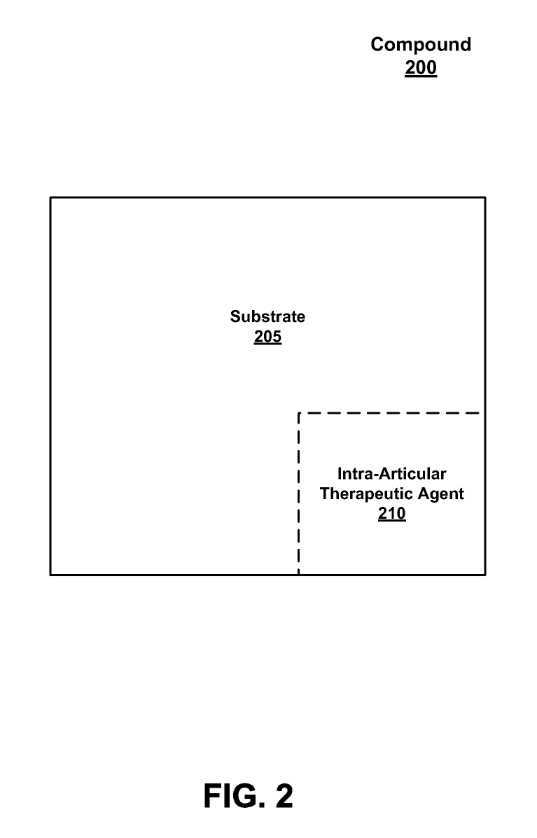

With reference now to FIG. 2, a block diagram of an intra-articular therapeutic agent delivery compound in accordance with embodiments of the present technology. In particular, FIG. 2 illustrates compound 200 composed of substrate 205 and intra-articular therapeutic agent 210. It should be appreciated that FIG. 2 is present for purposes of describing an intra-articular therapeutic agent delivery compound and is not meant to depict a specific molecular structure or binding. Nor is FIG. 2 meant to limit the present technology to the diagram shown.

In one embodiment, substrate 205 may be composed of acrylonitrile butadiene styrene (ABS) resin or polymer such as PGLA-Novador. In one embodiment, compound 200 is applied to an intra-articular site such as intra-articular site 110 of FIG. 1. In one embodiment, compound 200 allows intra-articular therapeutic agents 210 to separate from substrate 205 over a period of time and be distributed to the intra-articular site. In this manner, intra-articular therapeutic agent 210 is released to the intra-articular site in a controlled manner and may take effect at time later than the application time. In one embodiment, intra-articular therapeutic agents 210 comprises more than one entity that are not all released at once, but are released one at a time over a time period. In one embodiment, intra-articular therapeutic agent 210 represents a plurality of drugs or medications.

It should be appreciated that compound 200 may be formed in any number of shapes, such as spherical, cylindrical, cubical, conical, ellipsoidical, biconvex, hemispherical or near-hemispherical etc. By “near-hemispherical”, it is meant that one face of the device is substantially flat, shallow convex or shallow concave, and the opposite face is deeply convex (i.e., the deeply convex face has a greater radius of curvature than the shallow convex, shallow concave, or substantially flat face). In one embodiment, the shape of substrate 205 encompasses or surrounds intra-articular therapeutic agent 210. In one embodiment, intra-articular therapeutic agent 210 encompasses or surrounds substrate 205.

In various embodiments, the size of compound 200 is generally small such that the devices can be implanted in the fairly small openings in the human body. For example, when the device is cylindrical, it may be about 3-7 millimeters in height and about 0.5 to 4 millimeters in diameter. The volume of the device is such that the device holds sufficient amount of the drug to provide a continuous delivery over the implant's long delivery period, e.g., several weeks, months, or even longer, i.e., up to 2 or more years. The volume needed thus depends on characteristics such as drug solubility, drug delivery rate, period of delivery, drug's half life, etc. Once implanted, the device gives a continuous delivery of the drug to intra-articular sites without requiring additional invasive penetrations into these regions.

It should be appreciated that compound 200 may be composed of any number of materials including various biocompatible substantially impermeable polymeric compositions employed in preparing substrate 205. Some relevant factors to be considered in choosing a polymeric composition include: compatibility of the polymer with the biological environment of the implant, compatibility of the drug with the polymer, ease of manufacture, a half-life in the physiological environment of at least several days, no significant enhancement of the viscosity of the vitreous, and the desired rate of release of the drug. Depending on the relative importance of these characteristics, the compositions can be varied. Several such polymers and their methods of preparation are well-known in the art. See, for example, U.S. Pat. Nos. 6,331,313; 4,304,765; 4,668,506; 4,959,217; 4,144,317; and 5,824,074.

In one embodiment, substrate 205 comprises microspheres of biodegradable polymers. Such microspheres may be prepared with polymers of poly(lactic acid) or copolymers of glycolic acid and lactic acid. In such an embodiment, the mass of the microspheres, after implementation to the intra-articular site, will gradually become smaller and finally disappear thus releasing intra-articular therapeutic agent 210.

The polymers of interest may be homopolymers, copolymers, straight, branched-chain, or cross-linked derivatives. Some exemplary polymers include: polycarbamates or polyureas, cross-linked poly(vinyl acetate) and the like, ethylene-vinyl ester copolymers having an ester content of 4 to 80% such as ethylene-vinyl acetate (EVA) copolymer, ethylene-vinyl hexanoate copolymer, ethylene-vinyl propionate copolymer, ethylene-vinyl butyrate copolymer, ethylene-vinyl pentantoate copolymer, ethylene-vinyl trimethyl acetate copolymer, ethylene-vinyl diethyl acetate copolymer, ethylene-vinyl 3-methyl butanoate copolymer, ethylene-vinyl 3-3-dimethyl butanoate copolymer, and ethylene-vinyl benzoate copolymer, or a mixture thereof.

Additional examples include polymers such as: poly(methylmethacrylate), poly(butylnethacrylate), plasticized poly(vinylchloride), plasticized poly(amides), plasticized nylon, plasticized soft nylon, plasticized poly(ethylene terephthalate), natural rubber, silicone, poly(isoprene), poly(isobutylene), poly(butadiene), poly(ethylene), poly(tetrafluoroethylene), poly(vinylidene chloride), poly(acrylonitrile, cross-linked poly(vinylpyrrolidone), chlorinated poly(ethylene), poly(trifluorochloroethylene), poly(ethylene chlorotrifluoroethylene), poly(tetrafluoroethylene), poly(ethylene tetrafluoroethylene), poly(4,4′-isopropylidene diphenylene carbonate), polyurethane, poly(perfluoroalkoxy), poly(vinylidenefluoride), vinylidene chloride-acrylonitrile copolymer, vinyl chloride-diethyl fumarate copolymer, silicone, silicone rubbers (of medical grade such as Silastic® Medical Grade ETR Elastomer Q7-4750 or Dow Corning® MDX 4-4210 Medical Grade Elastomer); and cross-linked copolymers of polydimethylsilane silicone polymers.

Some further examples of polymers include: poly(dimethylsiloxanes), ethylene-propylene rubber, silicone-carbonate copolymers, vinylidene chloride-vinyl chloride copolymer, vinyl chloride-acrylonitrile copolymer, vinylidene chloride-acrylonitrile copolymer, poly(olefins), poly(vinyl-olefins), poly(styrene), poly(halo-olefins), poly(vinyls) such as polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, poly(acrylate), poly(methacrylate), poly(oxides), poly(esters), poly(amides), and poly(carbonates), or a mixture thereof.

In some aspects, the devices may be biodegradable wherein the outer layer degrades after the drug has been released for the desired duration. The biodegradable polymeric compositions may comprise organic esters or ethers, which when degraded result in physiologically acceptable degradation products, including the monomers. Anhydrides, amides, orthoesters or the like, by themselves or in combination with other monomers, may find use. The polymers may be addition or condensation polymers, cross-linked or non-cross-linked. For the most part, besides carbon and hydrogen, the polymers will include oxygen and nitrogen, particularly oxygen. The oxygen may be present as oxy, e.g., hydroxy or ether, carbonyl, e.g., non-oxo-carbonyl, such as carboxylic acid ester, and the like. The nitrogen may be present as amide, cyano and amino. In some aspects, the polymer is polytetrafluoroethylene, (commercially known as Teflon®), ethyl vinyl alcohol or ethylene vinyl acetate.

Some examples of biodegradable polymers useful in the present invention include: hydroxyaliphatic carboxylic acids, either homo- or copolymers, such as polylactic acid, polyglycolic acid, polylactic glycolic acid; polysaccharides such as cellulose or cellulose derivatives such as ethyl cellulose, cross-linked or uncross-linked sodium carboxymethyl cellulose, sodium carboxymethylcellulose starch, cellulose ethers, cellulose esters such as cellulose acetate, cellulose acetate phthallate, hydroxypropylmethyl cellulose phthallate and calcium alginate, polypropylene, polybutyrates, polycarbonate, acrylate polymers such as polymethacrylates, polyanhydrides, polyvalerates, polycaprolactones such as poly-.epsilon.-caprolactone, polydimethylsiloxane, polyamides, polyvinylpyrollidone, polyvinylalcohol phthallate, waxes such as paraffin wax and white beeswax, natural oils, shellac, zein, or a mixture thereof.

Suitable biodegradable polymeric compositions can be easily obtained since the decomposition rate of biodegradable polymers can be varied by chemical modification and/or by varying the component ratios and/or by varying the molecular weight. In case of certain polymers, isomerism can give rise to polymers with distinct characteristics. For example, by using the L-lactate, a slowly eroding polymer is achieved, while erosion is substantially enhanced with the lactate racemate.

It should be appreciated that intra-articular therapeutic agent 210 may be, but is not limited to, a conventional drug or medication, a plurality of drugs, an anti-inflammatory drug, a non-steroidal anti-inflammatory drug, an analgesic drug, or a combination thereof. In one embodiment, substrate 205 dissolves in a timely fashion to break the bond(s) between substrate 205 intra-articular therapeutic agent 210. Once the bond(s) are broken, intra-articular therapeutic agent 210 may begin to carry out its intended purposes at the intra-articular site.

Intra-articular therapeutic agent 210 comprises the drug or drugs to be delivered. It should be appreciated that there is no limit to the ratio of the amount of intra-articular therapeutic agent 210 to the amount substrate 205. Thus, the ratio of intra-articular therapeutic agent 210 to substrate 205 is dictated by the desired time span, the release rate, and the efficacy of the drug. For example, intra-articular therapeutic agent 210 may be from about 1 to 80, and in some aspects, from about 20 to 40 weight percent of substrate 205.

It should be appreciated that intra-articular therapeutic agent 210 can be comprised of a dry powder, particles, granules, liquid or as a compressed solid. Intra-articular therapeutic agent 210 may also be present as a solution or be dispersed in a polymer matrix. The polymers used in compound 200 with intra-articular therapeutic agent 210 are bio-compatible with body tissues and body fluids and can be biodegradable or substantially insoluble in the body fluids. Any of the above-described biocompatible polymer compositions can be used to prepare compound 200. The amount of polymer in the core may be from about 0% to 80 wt % by weight. These polymers are commercially available and methods for preparing polymer matrices are well-known in the art. See, for example, U.S. Pat. No. 5,882,682.

### Operation

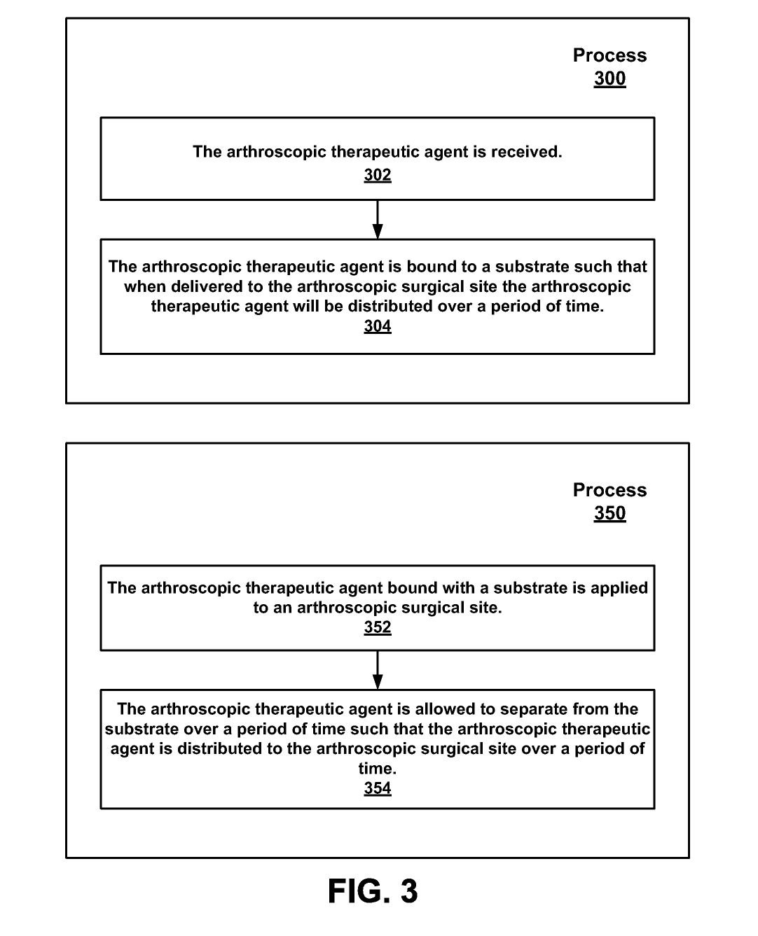

With reference now to process 300 of FIG. 3, a flowchart for forming an intra-articular therapeutic agent delivery compound for controlled delivery of an intra-articular therapeutic agent to an intra-articular site in accordance with embodiments of the present technology. In one embodiment, process 300 is method for manufacturing an intra-articular therapeutic agent delivery compound. In one embodiment, intra-articular site is an arthroscopic surgical site. In one embodiment, process 300 is used to form compound 200 of FIG. 2.

It should be appreciated that process 300 may be carried out using some or all of the steps shown in process 300. Process 300 depicts an order of steps for purposes of the illustration; the order shown in process 300 should not be construed to limit the present technology.

At 302, the intra-articular therapeutic agent is received. In one embodiment, a plurality of intra-articular therapeutic agents may be received. In one embodiment, more than one type of intra-articular therapeutic agents are received. Intra-articular therapeutic agents may be, but are not limited to, a conventional drug or medication, a plurality of drugs, an anti-inflammatory drug, a non-steroidal anti-inflammatory drug, an analgesic drug, or a combination thereof.

At 304, the intra-articular therapeutic agent is bound to a substrate such that when delivered to the intra-articular site the intra-articular therapeutic agent will be distributed over a period of time. In one embodiment, the substrate may be composed of acrylonitrile butadiene styrene (ABS) resin. In one embodiment, the substrate may be composed of a polymer. In one embodiment, the intra-articular therapeutic agent is bound to a substrate in a manner such that when injected into an intra-articular site, the bonds will be broken over a period of time.

With reference now to process 350 of FIG. 3, a flowchart for controlled delivery of an intra-articular therapeutic agent to an intra-articular site an event in accordance with embodiments of the present technology. It should be appreciated that process 350 may be carried out using some or all of the steps shown in process 350. Process 350 depicts an order of steps for purposes of the illustration; the order shown in process 350 should not be construed to limit the present technology. In one embodiment, intra-articular site is an arthroscopic surgical site. In one embodiment, process 350 takes place at intra-articular site 110 of FIG. 1.

At 352, the intra-articular therapeutic agent bound with a substrate is applied to an intra-articular site. In one embodiment, the application is carried out using a syringe to inject an intra-articular therapeutic agent delivery compound to the intra-articular site. In one embodiment, the intra-articular site is bathed in the intra-articular therapeutic agent delivery compound during a surgical procedure. In one embodiment, the intra-articular therapeutic agent delivery compound is combined with another fluid and applied to the site. In one embodiment, the application occurs peri-operatively. In one embodiment, the application occurs during a procedure performed at the intra-articular site.

At 354, the intra-articular therapeutic agent is allowed to separate from the substrate over a period of time such that the intra-articular therapeutic agent is distributed to the intra-articular site over a period of time. In one embodiment, such separation occurs when the bonds between the intra-articular therapeutic agent and the substrate break down. In one embodiment, such breakdown occurs because the bond is designed to only last for a specified time period. In one embodiment, such breakdown occurs when the intra-articular therapeutic agent delivery compound mixes with the natural chemistry of the intra-articular site.

Embodiments of the present technology are thus described. While the present technology has been described in particular embodiments, it should be appreciated that the present technology should not be construed as limited to these embodiments alone, but rather construed according to the following claims.

What is claimed is:

1. An intra-articular therapeutic agent delivery compound for controlled delivery of an intra-articular therapeutic agent to an intra-articular site, said compound comprising:
an intra-articular therapeutic agent; and
a substrate configured to bind with said intra-articular therapeutic agent, to be delivered to said intra-articular site, and to be distributed over a period of time thus releasing said intra-articular therapeutic agent.

2. The intra-articular therapeutic agent delivery compound of claim 1 wherein said intra-articular therapeutic agent is a plurality of drugs.

3. The intra-articular therapeutic agent delivery compound of claim 1 wherein said intra-articular therapeutic agent is an anti-inflammatory drug.

4. The intra-articular therapeutic agent delivery compound of claim 1 wherein said intra-articular therapeutic agent is a non-steroidal anti-inflammatory drug.

5. The intra-articular therapeutic agent delivery compound of claim 1 wherein said intra-articular therapeutic agent is an analgesic drug.

6. The intra-articular therapeutic agent delivery compound of claim 1 wherein said substrate is a biocompatible polymeric composition.

7. The intra-articular therapeutic agent delivery compound of claim 1 wherein said intra-articular therapeutic agent delivery compound is a liquid form.

8. The intra-articular therapeutic agent delivery compound of claim 1 wherein said intra-articular site is an arthroscopic surgical site.

9. The intra-articular therapeutic agent delivery compound of claim 1 wherein said intra-articular therapeutic agent and said substrate are configured to be delivered to said intra-articular site via injection with a syringe.

10. A method for forming an intra-articular therapeutic agent delivery compound for controlled delivery of an intra-articular therapeutic agent to an intra-articular site, said method comprising:
receiving said intra-articular therapeutic agent; and
binding said intra-articular therapeutic agent to a substrate such that when delivered to said intra-articular site said intra-articular therapeutic agent will be distributed over a period of time.

11. The method of claim 10 wherein said receiving said intra-articular therapeutic agent comprises receiving a plurality of drugs.

12. The method of claim 10 wherein said receiving said intra-articular therapeutic agent comprises receiving an anti-inflammatory drug.

13. The method of claim 10 wherein said receiving said intra-articular therapeutic agent comprises receiving a non-steroidal anti-inflammatory drug.

14. The method of claim 10 wherein said receiving said intra-articular therapeutic agent comprises receiving an analgesic drug.

15. The method of claim 10 wherein said binding said intra-articular therapeutic agent to said substrate comprises binding said intra-articular therapeutic agent to a biocompatible polymeric composition.

16. A method for controlled delivery of an intra-articular therapeutic agent to an intra-articular site, said method comprising:
applying said intra-articular therapeutic agent bound with a substrate to an intra-articular site; and
allowing said intra-articular therapeutic agent to separate from said substrate over a period of time such that said intra-articular therapeutic agent is distributed to said intra-articular site over a period of time.

17. The method of claim 16 wherein said applying further comprises bathing said intra-articular site with said intra-articular therapeutic agent bound with a substrate during a procedure.

18. The method of claim 16 wherein said applying occurs peri-operatively.

19. The method of claim 16 wherein said applying occurs during a procedure performed at said intra-articular site.

20. The method of claim 16 wherein said applying further comprises injecting said intra-articular therapeutic agent bound with said substrate into said intra-articular site using a syringe.
